(19) United States
(12) Patent Application Publication
(10) Pub. No.: US 2019/0111064 A1
(43) Pub. Date: Apr. 18, 2019

(54) STATIN + VITAMIN D COMBINATION DRUG AND METHOD OF USE

(71) Applicant: Interstice Therapeutics, LLC, Bedminster, NJ (US)

(72) Inventor: Andrew Isaacs, Bedminster, NJ (US)

(73) Assignee: Interstice Therapeutics, LLC, Bedminster, NJ (US)

(21) Appl. No.: 16/160,432

(22) Filed: Oct. 15, 2018

Related U.S. Application Data
(60) Provisional application No. 62/573,826, filed on Oct. 18, 2017.

(51) Int. Cl. A61K 31/593; A61P 21/00; A61P 9/00; A61P 3/06; A61K 31/592; A61K 9/51

(52) U.S. Cl. CPC A61K 31/593; A61P 21/00; A61P 9/00; A61K 31/366; A61K 31/592; A61K 9/5107; A61K 2300/00; A61P 3/06

(57) ABSTRACT

A pharmaceutical composition and method reduces statin-related side effects, including myalgia, myositis, myopathy, and myonecrosis. The composition comprises at least one HMG-CoA reductase inhibitor (statin) and vitamin D. The composition may comprise one or more excipient. The pharmaceutical composition is adapted to decrease side effects of said HMG-CoA reductase inhibitors. The excipient may comprise one or more excipient delivery agent providing immediate release, sustained release, extended release and/or combination thereof of said HMG-CoA and/or vitamin D. The amount of the vitamin D may be fixed or variable. A method of treating hyperlipidemia is provided comprising an orally administered pharmaceutical composition having at least one HMG-CoA reductase inhibitor and vitamin D. Preferably, the pharmaceutical composition is administered once per day, via oral, nonoral, transdermal and/or injection drug delivery.

This application claims the benefit of U.S. Provisional Patent Application No. 62/573826 filed Oct. 18, 2017, entitled “Statin+Vitamin D Combination Drug,” the disclosure of which is hereby incorporated in its entirety by reference thereto.

### 1. Field of the Invention

This disclosure relates to lipid-lowering agents and methods of treatment, and more particularly to improved compositions of HMG-CoA reductase inhibitors (statins) and Vitamin D for decreasing side effects associated with treating hyperlipidemia.

### 2. BACKGROUND

Cholesterol is a waxy, fat-like substance found in all cells of the body. The human body needs some cholesterol to make hormones, vitamin D, and substances that help digest foods. Human bodies typically produce all the cholesterol it that is necessary for these functions. However, cholesterol also is found in some foods.

Hyperlipidemia is a medical term that refers to a condition when blood has too many lipids (fats) in it, i.e., cholesterol and triglycerides. In hypercholesterolemia, there's too much LDL (bad) cholesterol in the blood. This increases fatty deposits in arteries and the risk of blockages.

People who have high blood cholesterol have a greater chance of getting coronary heart disease, also called coronary artery disease. The higher the level of LDL cholesterol in blood, the greater the chance is of getting heart disease. The higher the level of HDL cholesterol in blood, the lower the chance is of getting heart disease.

Coronary heart disease is a condition in which plaque builds up inside the coronary (heart) arteries. Plaque is made up of cholesterol, fat, calcium, and other substances found in the blood. When plaque builds up in the arteries, the condition is called atherosclerosis. Over time, plaque hardens and narrows coronary arteries, which limits the flow of oxygen-rich blood to the heart. Eventually, an area of plaque can rupture (break open) and can cause a blood clot to form on the surface of the plaque. If the clot becomes large enough, it can mostly or completely block blood flow through a coronary artery. If the flow of oxygen-rich blood to your heart muscle is reduced or blocked, angina (chest pain) or a heart attack may occur.

According to the National Institute for Health (NIH), the approximate percentage of the population of the United States that has high cholesterol by decade-age bracket is as follows: 22% for 20's, 38% for 30's, 50% for 40's, and 62% for 50's, and nearly 1 in every 2 women have either borderline/high cholesterol. Therefore, this is a serious health condition across a very broad patient spectrum.

High blood cholesterol is treated with Total Lifestyle Changes (TLC) and cholesterol-lowering medicines, with the main goal to lower low-density lipoprotein (LDL) cholesterol level enough to the reduced risk for coronary heart disease, heart attack, and other related health problems; the risk for heart disease and heart attack goes up as your LDL cholesterol level rises and your number of heart disease risk factors increases.

The cholesterol-lowering medicines can help control high blood cholesterol, but they don't cure it. Thus, patients prescribed these medicines must continue taking them to keep cholesterol levels in the recommended range. The five major classes of cholesterol-lowering medicines are statins, bile acid sequestrants, nicotinic acid, fibrates, and ezetimibe.

Statins are recommended for most patients because they're the only cholesterol-lowering drug class that's been directly associated with reducing the risk of a heart attack or stroke. Statins as a class of drugs, work in the liver to prevent cholesterol from forming by reducing the amount of cholesterol circulating in the blood.

Statins are most effective at lowering LDL (bad) cholesterol and they also help lower triglycerides (blood fats) and raise HDL (good) cholesterol. Examples of statins now available in the U.S. include, for nonlimiting example: Atorvastatin, such as that associated with the trade name Lipitor®; Fluvastatin, such as that associated with the trade name Lescol®; Lovastatin, such as that associated with the trade names Mevacor® and Altoprev™; Pravastatin, such as that associated with the trade name Pravachol®; Rosuvastatin Calcium, such as that associated with the trade name Crestor®; and Simvastatin, such as that associated with the trade name Zocor®. Statins are also found in combination medications, including for nonlimiting example: lovastatin+niacin, such as that associated with the trade name Advicor®; atorvastatin+amlodipine, such as that associated with the trade name Caduet®; and simvastatin+ezetimibe, such as that associated with the trade name Vytorin™.

While statins have proven effective in safely lowering LDL cholesterol, statin-related side effects have become a major concern. Specifically, side effects including myalgia, myositis, myopathy, and/or myonecrosis are relatively prevalent. Myalgia, myositis, myopathy and/or myonecroses pain directly related to taking statin drugs causes many patients to stop taking statins. The biological mechanism for statin-related myalgia is unknown. Some data indicates that vitamin D levels may play a role. A vitamin D deficiency itself is known to produce symptoms approximating statin-related myalgia. Some studies suggest that while some statin-treated patients had increased odds of myalgia, statin users with higher levels of vitamin D did not have an increased risk for myalgia when compared to non-statin users.

Statins are generally considered safe for most users, however according to published articles, approximately 2% to 20% of patients experience statin-related myalgia. Many patients do not report myalgia symptoms but stop taking the drug due to muscle-related pain. Statin-related myalgia may feel like a dull ache initially, but it can be severe. Some patients describe the pain as aches, pain and fatigue typically associated with a severe flu. Unfortunately, patients often have to resort to discontinuing statin therapy to eliminate the intolerable side effects, thus assuming the risks associated with their original diagnosis of hyperlipidemia. In addition, there is also an additional unreported population that refuses, or cannot even start, statin therapy due to myalgia concerns.

Vitamin D is a fat-soluble vitamin that is naturally present in very few foods, added to others, and available as a dietary supplement. Vitamin D is made up of a group of fat-soluble secosteroids responsible for increasing intestinal absorption of calcium, magnesium, and phosphate, and multiple other biological effects, with the most important compounds in this group being vitamin D₃ (cholecalciferol) and vitamin D₂ (ergocalciferol). It is also produced endogenously when ultraviolet rays from sunlight strike the skin and trigger vitamin D synthesis. Vitamin D obtained from sun exposure, food, and supplements.

According to the NIH, vitamin D promotes calcium absorption in the gut and maintains adequate serum calcium and phosphate concentrations to enable normal mineralization of bone and to prevent hypocalcemic (low blood calcium) tetany (muscular spasms). It is also needed for bone growth and bone remodeling by osteoblasts and osteoclasts. Without sufficient vitamin D, bones can become thin, brittle, or misshapen.

Obtaining sufficient vitamin D from natural food sources alone is difficult. For many people, consuming vitamin D-fortified foods and, arguably, being exposed to some sunlight are essential for maintaining a healthy vitamin D status. In some groups, dietary supplements might be required to meet the daily need for vitamin D. Therefore, providing vitamin D as a supplement to those that are at high risk for coronary artery disease would have incremental benefits as indicated above, including the potential to minimize or eliminate myalgia related pain to ensure adherence and persistence to life saving medications such as statins.

Various combinations of statins have been proposed. These include, for example:

U.S. Statutory Invention registration H1286, published Feb. 1, 1994, discusses various combinations of HMG CoA reductase inhibitors with agents which lower lipids via other mechanisms. Niacin, or nicotinic acid, is among the agents discussed.

U.S. Pat. No. 5,260,305 to Dennick discloses a pharmaceutical combination which includes an inhibitor of the enzyme 3-hydroxy-3-methylglutaryl coenzyme A (HMG CoA) reductase, which is pravastatin and a pharmaceutical which reduces serum cholesterol and/or inhibits cholesterol biosynthesis by a mechanism other than inhibiting production of the enzyme HMG CoA reductase, namely, nicotinic acid (niacin) or related acid. A method for reducing serum cholesterol or inhibiting formation of or treating atherosclerosis is disclosed using the above combination without causing drug-induced myopathy or rhabdomyolysis, is also provided.

U.S. Publication Number WO1999017774 to Nolan, discloses lipid-lowering dosage units, to be used once a day, to produce minimal side effects. The dosage units contain a combination of microspheres formulated to co-deliver a HMG-CoA reductase inhibitor component and a niacin component.

U.S. Pat. No. 5,157,025 relates to a method for lowering serum cholesterol and thereby inhibiting fatty streak lesions of atherosclerosis by administering to a patient a phosphorus-containing ACE inhibitor, such as fosinopril or ceronapril, alone or in combination with a cholesterol-lowering drug, such as pravastatin.

While combinations of lipid-lowering agents are known, there is still a need for combinations in which each of the types of active components therein have been specifically formulated in order to optimize their release properties and, thereby, significantly minimize the likelihood that they will produce unwanted side effects when consumed. At the same time, there is a need for a dosage regimen which is more convenient to the patient than those currently available in the art. There is further a need in the art to provide a statin and vitamin D composition that effectively releases vitamin D in conjunction with a statin in order to reduce the risk of side effects, indulging myalgia, myositis, myopathy, and/or myonecrosis.

### SUMMARY OF THE INVENTION

The present invention provides drug compositions, methods of manufacturing same, and methods of use in treating side effects associated with the use of statins. The subject composition provides dosage delivery of satin with vitamin D so that the patient takes both active ingredients at the same time in one pill, injection, or dosage. By providing vitamin D dosage with statin dosage within the same pill, capsule, injection, etc., the combination of the active components are specially formulated to provide a synergistic effect that significantly minimizes side effects, including for nonlimiting example, myalgia, myositis, myopathy, and/or myonecrosis. At the same time, the subject composition and methods provide a dosage regimen which is more convenient to the patient than those currently available in the art.

The subject drug composition and method is made combining statin drug products with vitamin D. The subject composition and method is provided for use in treating patients with high cholesterol by reducing the levels of LDL and who experience statin-related myalgia, myositis, myopathy, and/or myonecrosis as a result of standard statin drug treatments. The subject combination of vitamin D and statin drug composition reduces pain and feelings of malaise associated with statin-related myalgia, myositis, myopathy, and/or myonecrosis.

In a first aspect of the invention, a pharmaceutical composition is provided comprising at least one HMG-CoA reductase inhibitor (statin); vitamin D; and one or more excipient, whereby the pharmaceutical composition is adapted to decrease side effects of the HMG-CoA reductase inhibitor.

In another aspect of the invention, there is provided a pharmaceutical composition comprising at least one HMG-CoA reductase inhibitor and vitamin D.

Another aspect of the invention provides a method of treating hyperlipidemia comprising a pharmaceutical composition comprising at least one HMG-CoA reductase inhibitor and vitamin D, said pharmaceutical composition being administered by oral, non-oral, transdermal and/or injection drug delivery. Preferably, the pharmaceutical composition is administered once per day.

### DETAILED DESCRIPTION OF THE INVENTION

It is desirable to improve upon HMG-CoA reductase inhibitors (statins) drug compositions and methods for the treatment of hyperlipidemia or high cholesterol by providing a composition having statins and Vitamin D for decreasing side effects. The invention comprises embodiments of a statin-vitamin D combination drug formulations. Drug delivery of the subject composition includes conventional drug delivery of statins, and particularly includes oral, nonoral, transdermal and/or injection drug delivery and/or combination thereof. An aspect of this invention is that statin-vitamin D combination drugs show a surprising and reproducible benefit of helping relieve the pain experienced by patients suffering from statin-related myalgia, myositis, myopathy, and/or myonecrosis. Another aspect of the invention concerns convenient delivery methods to ensure compliance/adherence to taking statin medications. Another aspect of this invention concerns administration of appropriate relative amounts of a statin and vitamin D in a combination by way of oral, nonoral, transdermal and/or injection drug delivery to maximize efficacy but without unwanted side effects, like statin-related myalgia, myositis, myopathy, and/or myonecrosis. A further aspect of this invention concerns the specific rate of individual release of the statin and vitamin D into the body. The rate of release of either the statin or vitamin D can be independent of one another. For example, the statin may be immediately released into the body, whereas vitamin D can be released slowly, over time, in an extended manner. Either or both of the statin and vitamin D can be formulated to be for immediate release, extended release, or any combination of extended and immediate release. Preferably, the intended patient of the subject composition is found, for example via blood work, to have deficient and/or low levels of Vitamin D. Deficient or low levels being levels ≤16 ng/ml. Once a sufficient vitamin D target level is reached (i.e. blood testing shows that the levels are ≥ to 16 ng/ml [or recommended levels] the method of treatment utilizing the subject composition may be stopped. However, the subject combination provides general health benefits, even for those patients who are not suffering from myalgia/side effects from statins, and therefore it is not required that the patient exhibit low level of vitamin D and/or stop taking the subject composition even when target Vitamin D levels are achieved.

Vitamin D and most statin APIs are lipophilic molecules, so they often have poor bioavailability in vivo. Dissolution of the two lipophilic molecules and absorption through the GI tract can be both erratic and incomplete. Although some studies suggest that statin users with higher levels of vitamin D did not have an increased risk for myalgia, myositis, myopathy, and/or myonecrosis as compared to non-statin users, the role vitamin D plays in myalgia is unknown. On information and belief, without being bound by theory, it is believed that delivery of high levels of vitamin D contemporaneously with delivery of statins can reduce myalgia, myositis, myopathy, and/or myonecrosis side effects associated with statin therapy. The subject composition may be prescribed or taken by both patients found to be deficient or have low levels of vitamin D (i.e. ≤16 ng/ml), as well as patients having normal levels of Vitamin D to prevent potential statin related side effects, such as myalgia.

In particular, it is believed, without being bound by theory, that a delivery system that contemporaneously delivers vitamin D with statins confers therapeutic enhancement of the statin active ingredient(s) during dosage delivery, decreasing side effects, particularly myalgia. The composition provides release of Vitamin D, either in a large initial dosage and/or throughout the dosage release of the statin in the body to effectively reduce myalgia, myositis, myopathy, and/or myonecrosis. Without being bound by theory, it is believed that combining a statin drug's delivery with Vitamin D delivery not only addresses possible Vitamin D deficiencies but also results in a synergistic effect on the body.

Taking high levels of vitamin D along with statins may reduce the intolerable myalgia, myositis, myopathy, and/or myonecrosis side effects associated with statin therapy, which often causes up to 20% of patients to stop taking statins. In addition, there is also an additional unreported population that does not even start statin therapy due to myalgia, myositis, myopathy, and/or myonecrosis concerns. A combination unit of the two classes of drugs is now proposed with a single, daily dosing requirement, that may eliminate most if not all myalgia, myositis, myopathy, and/or myonecrosis side effects. In this way, a much-improved patient compliance record can be established and avoid the known non-compliance issues associated with taking multiple/separate oral pills. It is noted that the subject statin+Vitamin D composition may be delivered oral, nonoral, transdermal and/or injection. The new drug dosage unit is thus a considerable advancement over either drug alone, as well as over any presently known combination of the two classes of drugs. As hereinafter described, the daily amount of vitamin

D and the release characteristics of both classes of drugs may be modified to provide both the appropriate amount of vitamin D myalgia, myositis, myopathy, and/or myonecrosis prevention and lipid-lowering action to allow for the adaptation of the combination into a once-daily single unit dosage.

A combination unit of the statins and vitamin D is provided by way of the subject invention providing a single, daily dosing requirement that may eliminate most if not all myalgia, myositis, myopathy, and/or myonecrosis side effects. In this way, a much-improved patient compliance record can be established, thereby avoiding the known non-compliance issues associated with taking multiple/separate oral pills. The dosage unit is thus a considerable advancement over either drug alone, as well as over any presently known combinations of the two classes of drugs. As hereinafter described, the daily amount of vitamin D and the release characteristics of both classes of drugs may be modified to provide both the appropriate amount of vitamin D myalgia prevention and lipid-lowering action to allow for the adaptation of the combination into a once-daily single unit dosage. The vitamin D of the subject composition may be an amount in the composition equal to upper limits for Vitamin D in order align with recommended max daily toxicity limits. While combinations of lipid-lowering agents are known, the present invention addresses a need for combinations in which each of the types of active components therein have been specifically formulated in order to optimize their release properties and, thereby, significantly minimize the likelihood that they will produce unwanted side effects when consumed. At the same time, the present invention addresses the need for a dosage regimen, which is more convenient to the patient than those currently available in the art.

This invention comprises embodiments of a statin-vitamin D combination oral, nonoral, transdermal and/or injection drug formulation. An aspect of this invention is that statin-vitamin D combination oral drugs show a surprising and reproducible benefit of helping relieve the pain experienced by patients suffering from statin-related myalgia, myositis, myopathy, and/or myonecrosis. Another aspect of the invention concerns convenient delivery methods to ensure compliance/adherence to taking statin medications. Another aspect of this invention concerns administration of appropriate relative amounts of a statin and vitamin D in a combination oral drug to maximize efficacy but without unwanted side effects, like statin-related myalgia, myositis, myopathy, and/or myonecrosis. A further aspect of this invention concerns the specific rate of individual release of the statin and vitamin D into the body. The rate of release of either the statin or vitamin D can be independent of one another. For example, the statin may be immediately released into the body, whereas the vitamin D can be released slowly, over time, in an extended manner. Either or both the statin and vitamin D can be formulated to be for immediate release, extended release, or any combination of extended and immediate release.

In a first embodiment, the subject composition and method provides for Immediate Release Prescribed Amount of Statin and Immediate Release Fixed Vitamin D by patient Age. The combination of active ingredients (statin and vitamin D) may be the prescribed amount of an immediately released statin (the active ingredient varies by the amount needed to address the levels of HDL's) and an immediate released fixed daily recommended amount of Vitamin D by age (600 IU or 15 mcg for 18-70 years of age and 800 IU or 20 mcg for >70 years of age). Variations of the amount of Vitamin D may not be related to a patient's age.

Another aspect of the composition and method provides Immediate Release Prescribed Amount of Statin and Immediate Release Variable Prescribed Amount of Vitamin D Regardless of patient Age. The combination of active ingredients (statin and vitamin D) may be the prescribed amount of an immediately released statin (the active ingredient varies by the amount needed to address the levels of HDL's) and a prescribed amount of an immediate released Vitamin D; from the minimum daily requirements as indicated above (600 IU) to the upper limit of daily consumption limit (4000 IU or 100 mcg), regardless of age.

The composition and method may provide Immediate Release Prescribed Amount of Statin and Sustained Release Fixed Vitamin D by patient Age. The combination of active ingredients (statin and vitamin D) may be the prescribed amount of an immediately released statin (the active ingredient varies by the amount needed to address the levels of HDL's) and a sustained released fixed daily recommended amount of Vitamin D by age (600 IU or 15 mcg for 18-70 years of age and 800 IU or 20 mcg for >70 years of age).

In yet another aspect of the composition and method, Immediate Release Prescribed

Amount of Statin and Sustained Release Variable Prescribed Amount of Vitamin D Regardless of patient Age. The combination of active ingredients (statin and vitamin D) may be the prescribed amount of an immediately released statin (the active ingredient varies by the amount needed to address the levels of HDL's) and a prescribed amount of a sustained released Vitamin D; from the minimum daily requirements as indicated above (600 IU) to the upper limit of daily consumption limit (4000 IU or 100 mcg), regardless of age.

The composition and method may provide a Sustained Release Prescribed Amount of Statin and an Immediate Release Variable Prescribed Amount of Vitamin D by patient Age. The combination of active ingredients (statin and vitamin D) may be the prescribed amount of a sustained released statin (the active ingredient varies by the amount needed to address the levels of HDL's) and an immediate released fixed daily recommended amount of Vitamin D by age (600 IU or 15 mcg for 18-70 years of age and 800 IU or 20 mcg for >70 years of age).

The composition and method may provide Sustained Release Prescribed Amount of Statin and Immediate Release Variable Prescribed Amount of Vitamin D Regardless of patient Age. The combination of active ingredients (statin and vitamin D) may be the prescribed amount of a sustained release statin (the active ingredient varies by the amount needed to address the levels of HDL's) and an immediate release variable amount of vitamin D from the minimum daily requirements as indicated above (600 IU) to the upper limit of daily consumption limit (4000 IU or 100 mcg), regardless of age.

Sustained Release Prescribed Amount of Statin and Sustained Release Variable Prescribed Amount of Vitamin D by patient Age may be provided. The combination of active ingredients (statin and vitamin D) may be the prescribed amount of a sustained released statin (the active ingredient varies by the amount need to address the levels of HDL's) and a sustained released fixed daily recommended amount of Vitamin D by age (600 IU or 15 mcg for 18-70 years of age and 800 IU or 20 mcg for >70 years of age).

In another aspect of the invention, Sustained Release Prescribed Amount of Statin and Sustained Release Variable Prescribed Amount of Vitamin D Regardless of patient Age may be provided. The combination of active ingredients (statin and vitamin D) may be the prescribed amount of a sustained release statin (the active ingredient varies by the amount needed to address the levels of HDL's) and a sustained release variable amount of vitamin D from the minimum daily requirements as indicated above (600 IU) to the upper limit of daily consumption limit (4000 IU or 100 mcg), regardless of age.

The excipient may comprise one or more excipient delivery agents providing immediate release of the HMG-CoA and immediate release of the vitamin D. Alternatively, the pharmaceutical composition may comprise one or more excipient delivery agents providing immediate release of the HMG-CoA reductase inhibitor, and a sustained release of the vitamin D. The excipient may comprise one or more excipient delivery agents providing extended release of the HMG-CoA reductase inhibitor and an immediate release of the vitamin D. Optionally, the excipient delivery agent may provide extended release of the HMG-CoA reductase inhibitor and an extended release of the vitamin D. The amount of the vitamin D may be a fixed amount delivered by one or more of immediate release, sustained release and extended release. Preferably, the fixed amount of vitamin D is determined by a patient's age. In another embodiment, the amount of vitamin D is a variable amount delivered by one or more of immediate release, sustained release, and extended release. Preferably, the variable amount of vitamin D is not determined by the patient's age. The HMG-CoA reductase inhibitor may be delivered in dosage release of one or more of immediate release, sustained release, or a combination of immediate and sustained release. The vitamin D dosage may be delivered by immediate release, sustained release, or a combination of immediate and sustained release. The HMG-CoA reductase inhibitor is one or more of atorvastatin, lovastatin, pravastatin, fluvastatin, rosuvastatin calcium, and simvastatin. The pharmaceutical composition may comprise a nanoparticulate formulation with particle sizes less than 2500 nm, and wherein the excipient is water-solubilizing. The statin may be hydrophilic and excipients that allow for simultaneous absorption of hydrophobic and hydrophilic components in a GI tract.

Regarding toxicity, vitamin D can cause non-specific symptoms such as anorexia, weight loss, polyuria, and heart arrhythmias. More seriously, it can also raise blood levels of calcium, which leads to vascular, and tissue calcification, with subsequent damage to the heart, blood vessels, and kidneys.

The subject composition preferably comprises at least one statin, one or more Vitamin D group, preferably being Vitamin D3 and excipient. Although most data suggest a toxicity threshold for vitamin D of 10,000 to 40,000 IU/day and serum 25(OH)D levels of 500-600 nmol/L (200-240 ng/mL), the Food and Nutrition Board (FNB) at the Institute of Medicine of

The National Academies published guidelines for an upper limit (UL) of 4,000 international units (IU), which the FNB applied to children aged 9 and older and adults, with corresponding lower amounts for younger children. For example increased average daily consumption from 400 to 600 IU <70 years of age; increased average daily consumption from 600 to 800 IU >71 years of age.

Below is a dosage table for the subject composition and method of treatment. The table indicates statins associated with brand or tradenames and the associated active ingredient. In each instance, the statin is at a daily dosage as indicated, along with a maximum daily dosage. Vitamin D amounts in the statin+Vitamin D composition are set forth herein below in International Unit (IU), shown with Vitamin D immediate release drug delivery and sustained release drug delivery.

The Table II below indicates optional statin dosages with the dosages of Vitamin D from the table above applicable, for nonlimiting example:

Toxicity threshold for vitamin D of 10,000 to 40,000 IU/day and serum 25(OH)D levels of 500-600 nmol/L (200-240 ng/mL): below is a representative dosage indicator of the dosage amount in IU of Vitamin D, representative at dosage delivery points up to a maximum dosage of 40,000 IU, daily.

The subject composition preferably comprises the following dosage complex:

Composition=Excipient+(Max Statin Daily Dosage)+(Vitamin D Dosage)

wherein for Vitamin D Immediate Release Vitamin D, Dosage ranges from about 2,000 IU-40,000 IU; preferably, from about 4,000 IU-12,000 IU; and most preferably, from about 8,000 IU-12,000 IU.

wherein for Vitamin D Sustained Release Vitamin D, Dosage ranges from about 2,000 IU-30,000 IU; preferably, from about 4,000 IU-16,000 IU; and most preferably, from about 8,000 IU-12,000 IU.

Conversion from mg or mcg to IU, or vice versa: According to the Office of Dietary

Supplements and the Ultimate Pharmacy Calculations Guide: Vitamin D: 1 IU is the biological equivalent of 0.025 mcg cholecalciferol or ergocalciferol.

The Vitamin D Dosage amount may change predicated on a patient's blood work: for example, if a patient baseline blood work indicates a Vitamin D deficiency, a composition with a greater amount of Vitamin D closer to the maximum threshold [12,000; 30,000; 40,000 IU] may be prescribed. As the Vitamin D levels are measured at a later time, if test blood work indicates Vitamin D levels normal or above normal, a composition with a lower amount of

Vitamin D closer to the minimum threshold [˜4,000+] may be prescribed. Accordingly, while the statin dosage in the composition remains constant, dosage amounts of the Vitamin D may be adjusted based on Vitamin D levels in the patient's blood and/or if the patient is exhibiting side effects, a composition with a greater amount of Vitamin D may be prescribed.

Vitamin D₃ (cholecalciferol) may be utilized in the composition. Alternatively, Vitamin D₂ (ergocalciferol) may be utilized in the composition. The Vitamin D complex may include one or more of vitamin D₃ (cholecalciferol) and vitamin D₂ (ergocalciferol). Preferably, Vitamin D₃ is used in the subject composition.

In some embodiments of this invention, the bioavailability and potency of statin-vitamin D formulations is found to be better in nanoparticulate formulations with particle sizes less than 2500 nm. Such nanoparticle formulations can be achieved through lyophilizing, milling, high-pressure homogenization, solution enhanced dispersions, spray freezing, and solid dispersions. In other embodiments, the bioavailability of statin-vitamin D combination formulations can be improved through the use of water-solubilizing excipients such as PEGs, surfactants, and carriers.

In other embodiments, hydrophilic statin APIs can be combined with vitamin D. In these embodiments, formulations can include excipients that allow for the simultaneous absorption of hydrophobic and hydrophilic components in the GI tract. For example, cyclodextrins may help provide a sustained release oral formulation of statin-vitamin D, each of which can be absorbed by the GI tract in a controlled manner. For example, a combination of hydrophobic and hydrophilic cyclodextrin excipients, in appropriate ratios, can be combined in a statin-vitamin D formulation that allows for maximum absorption of both the statin and vitamin D.

Excipients herein include one or more flavor enhancers, colorants, antiadherents, binders, coatings, stabilizers, bulking agents, fillers, diluents and/or therapeutic enhancement agents, such as agents facilitating drug absorption, reducing viscosity, or enhancing solubility for drug delivery. The excipient may depend upon the route of administration and the dosage form, as well as the active ingredient and other factors. Binders may include Saccharides and their derivatives, Disaccharides: sucrose, lactose, Polysaccharides and their derivatives: starches, cellulose or modified cellulose such as microcrystalline cellulose and cellulose ethers such as hydroxypropyl cellulose (HPC); Sugar alcohols such as xylitol, sorbitol or mannitol; Protein: gelatin; Synthetic polymers: polyvinylpyrrolidone (PVP), polyethylene glycol (PEG). Solution binders are dissolved in a solvent (for example water or alcohol can be used in wet granulation processes). Examples include gelatin, cellulose, cellulose derivatives, polyvinylpyrrolidone, starch, sucrose and polyethylene glycol. Dry binders are added to the powder blend, either after a wet granulation step, or as part of a direct powder compression (DC) formula. Examples include cellulose, methyl cellulose, polyvinylpyrrolidone and polyethylene glycol. Coatings may include tablet coatings, cellulose ether hydroxypropyl methylcellulose (HPMC) film coatings, and other coating materials, including for example synthetic polymers, shellac, corn protein zein or other polysaccharides. Capsules coated with gelatin. Excipients including transdermal patches/and or surface are contemplated for administration wherein active ingredients, statin and Vitamin D, are delivered across the skin for systemic distribution. Transdermal patches include providing a medicated adhesive patch adapted to be placed on the skin to deliver the subject statin+Vitamin D composition dose of medication through the skin and into the bloodstream. Excipients provided for oral, nonoral, transdermal and/or injection drug delivery are contemplated, as well as other drug delivery methods providing the subject statin and vitamin D synergistic dosage combination to target vitamin D levels to mitigate statin side effects.

The excipient may comprise one or more Enterics to control the rate of drug release and determine where the drug will be released in the digestive tract. Materials used for enteric coatings may include fatty acids, waxes, shellac, plastics, and plant fibers. The excipient may comprise Disintegrants that expand and dissolve when wet causing the tablet to break apart in the digestive tract, releasing the active ingredients for absorption, ensuring that when the tablet is in contact with water, it rapidly breaks down into smaller fragments, facilitating dissolution. Examples of disintegrants contemplated include: Crosslinked polymers: crosslinked polyvinylpyrrolidone (crospovidone), crosslinked sodium carboxymethyl cellulose (croscarmellose sodium); modified starch sodium starch glycolate. The excipient may further comprise glidants, lubricants, preservatives, sorbents, sweeteners, vehicles/medium for conveying the active ingredients, including for example Petrolatum, dimethyl sulfoxide and mineral oil are common vehicles.

The following examples are presented to provide a more complete understanding of the invention. The specific techniques, conditions, materials, proportions and reported data set forth to illustrate the principles and practice of the invention are exemplary and should not be construed as limiting the scope of the invention.

Example 1: Immediate Release Prescribed Amount of Statin and Immediate Release Fixed Vitamin D by patient Age. The combination of active ingredients (statin and vitamin D) is the prescribed amount of an immediately released statin (the active ingredient varies by the amount needed to address the levels of HDL's) and an immediate released fixed daily recommended amount of Vitamin D by age (daily recommended consumption: 600 IU or 15 mcg for 18-70 years of age and 800 IU or 20 mcg for >70 years of age). Preferably, the subject composition utilizes a higher amount of Vitamin D than the daily recommended, up to 40000 IU,consumption yet falls within max toxicity guidelines. For example, the subject composition Vitamin D dosage is > the daily recommended dosage, yet < the maximum daily (max dosage based on toxicity via regulatory agencies). Statin Intolerance decrease—myalgia, myositis, myopathy, and/or myonecrosis side effects are reduced by at least 95% after 12 months.

Example 2: Immediate Release Prescribed Amount of Statin and Immediate Release Variable Prescribed Amount of Vitamin D Regardless of patient Age. The combination of active ingredients (statin and vitamin D) is the prescribed amount of an immediately released statin (the active ingredient varies by the amount needed to address the levels of HDL's) and a prescribed amount of an immediate released Vitamin D; from the minimum daily requirements as indicated above (600 IU) to the upper limit of daily consumption limit (40000 IU), regardless of age. Statin Intolerance decrease—myalgia side effects are reduced by at least 95% after 12 months.

Example 3: Immediate Release Prescribed Amount of Statin and Sustained Release Fixed Vitamin D by patient Age. The combination of active ingredients (statin and vitamin D) is the prescribed amount of an immediately released statin (the active ingredient varies by the amount needed to address the levels of HDL's) and a sustained released fixed daily recommended amount of Vitamin D by age (>600 IU or 15 mcg for 18-70 years of age and >800 IU or 20 mcg for >70 years of age—up to 40000 IU. Statin Intolerance decrease—myalgia, myositis, myopathy, and/or myonecrosis side effects are reduced by at least 95% after 12 months.

Example 4: Immediate Release Prescribed Amount of Statin and Sustained Release Variable Prescribed Amount of Vitamin D Regardless of patient Age. The combination of active ingredients (statin and vitamin D) is the prescribed amount of an immediately released statin (the active ingredient varies by the amount needed to address the levels of HDL's) and a prescribed amount of a sustained released Vitamin D; from the minimum daily requirements as indicated above (600 IU) to the upper limit of daily consumption limit (40000 IU), regardless of age. Improved Statin Intolerance—myalgia side effects are reduced by at least 95% after 12 months.

Example 5: Sustained Release Prescribed Amount of Statin and an Immediate Release Variable Prescribed Amount of Vitamin D by patient Age. The combination of active ingredients (statin and vitamin D) is the prescribed amount of a sustained released statin (the active ingredient varies by the amount needed to address the levels of HDL's) and an immediate released fixed daily recommended amount of Vitamin D by age (≥600 IU or 15 mcg for 18-70 years of age and ≥800 IU or 20 mcg for >70 years of age) up to 40000 IU. Statin Intolerance decrease—myalgia side effects are reduced by ≥95% after 12 months.

Example 6: Sustained Release Prescribed Amount of Statin and Immediate Release Variable Prescribed Amount of Vitamin D Regardless of patient Age. The combination of active ingredients (statin and vitamin D) is the prescribed amount of a sustained release statin (the active ingredient varies by the amount needed to address the levels of HDL's) and an immediate release variable amount of vitamin D from the minimum daily requirements as indicated above (600 IU) to the upper limit of daily consumption limit (40000 IU or 1000 mcg), regardless of age.

Example 7: Sustained Release Prescribed Amount of Statin and Sustained Release Variable Prescribed Amount of Vitamin D by patient Age. The combination of active ingredients (statin and vitamin D) is the prescribed amount of a sustained released statin (the active ingredient varies by the amount needed to address the levels of HDL's) and a sustained released fixed daily recommended amount of Vitamin D by age (≥600 IU or 15 mcg for 18-70 years of age and ≥800 IU or 20 mcg for >70 years of age) up to 40000 IU. Myalgia, myositis, myopathy, and/or myonecrosis side effects are reduced by ≥95% after 12 months.

Example 8: Sustained Release Prescribed Amount of Statin and Sustained Release Variable Prescribed Amount of Vitamin D Regardless of patient Age. The combination of active ingredients (statin and vitamin D) is the prescribed amount of a sustained release statin (the active ingredient varies by the amount needed to address the levels of HDL's) and a sustained release variable amount of vitamin D ≥ the minimum daily requirements. Myalgia side effects are reduced by ≥95% after 12 months.

Particular embodiments of the subject matter described in this specification have been described. Other embodiments are within the scope of the following claims. For example, the actions recited in the claims can be performed in a different order and still achieve desirable results, unless expressly noted otherwise.

Having thus described the invention in rather full detail, it will be understood that such detail need not be strictly adhered to, but that additional changes and modifications may suggest themselves to one skilled in the art, all falling within the scope of the invention as defined by the subjoined claims.

What is claimed is:

1. A pharmaceutical composition comprising:
(a) at least one HMG-CoA reductase inhibitor (statin);
(b) vitamin D; and
(c) one or more excipient;
whereby said pharmaceutical composition decreases side effects of said HMG-CoA reductase inhibitor.

2. The pharmaceutical composition as recited by claim 1, wherein said excipient comprises one or more excipient delivery agent providing immediate release of said HMG-CoA and immediate release of said vitamin D.

3. The pharmaceutical composition as recited by claim 1, wherein said excipient comprises one or more excipient delivery agent providing immediate release of said HMG-CoA reductase inhibitor and a sustained release of said vitamin D.

4. The pharmaceutical composition as recited by claim 1, wherein said excipient comprises one or more excipient delivery agent providing extended release of said HMG-CoA reductase inhibitor and an immediate release of said vitamin D.

5. The pharmaceutical composition as recited by claim 1, wherein said excipient comprises one or more excipient delivery agent providing extended release of said HMG-CoA reductase inhibitor and an extended release of said vitamin D.

6. The pharmaceutical composition as recited by claim 1, wherein said amount of said vitamin D is a fixed amount delivered by one or more of immediate release, sustained release and extended release.

7. The pharmaceutical composition as recited by claim 1, wherein said amount of said vitamin D is a variable amount delivered by one or more of immediate release, sustained release and extended release.

8. The pharmaceutical composition as recited by claim 1, wherein said side effect is myalgia.

9. The pharmaceutical composition as recited by claim 1, wherein said side effect is at least one of myalgia, myositis, myopathy, and myonecrosis.

10. The pharmaceutical composition as recited by claim 1, wherein said HMG-CoA reductase inhibitor is immediate release, sustained release, or a combination of immediate and sustained release.

11. The pharmaceutical composition as recited by claim 1 wherein said vitamin D is immediate release, sustained release, or a combination of immediate and sustained release.

12. The pharmaceutical composition as recited by claim 1, wherein said HMG-CoA reductase inhibitor is one or more of atorvastatin, lovastatin, pravastatin, fluvastatin, rosuvastatin calcium and simvastatin.

13. The pharmaceutical composition as recited by claim 1 comprising a nanoparticulate formulation with particle sizes less than 2500 nm, and wherein said excipient is water-solubilizing.

14. The pharmaceutical composition as recited by claim 1, wherein said statin is hydrophilic and excipients that allow for simultaneous absorption of hydrophobic and hydrophilic components in a GI tract.

15. The pharmaceutical composition as recited by claim 1, wherein said vitamin D is an amount ranging from about 600 IU to 40000 IU.

16. A pharmaceutical composition comprising at least one HMG-CoA reductase inhibitor and vitamin D.

17. A method of treating hyperlipidemia comprising a pharmaceutical composition comprising at least one HMG-CoA reductase inhibitor and vitamin D, said pharmaceutical composition being administered by an oral, non-oral, transdermal and/or injection delivery method.

18. The method as recited by claim 17 comprising at least one excipient.

19. The method as recited by claim 17, wherein said pharmaceutical composition is administered once per day.

20. The method as recited by claim 17, wherein the pharmaceutical composition reduces at least one of statin-related myalgia, myositis, myopathy, and myonecrosis.
